(19) United States
(12) Patent Application Publication
(10) Pub. No.: US 2021/0106263 A1
(43) Pub. Date: Apr. 15, 2021

(54) BEHAVIOR INDUCTION SYSTEM, BEHAVIOR INDUCTION METHOD, AND RECORDING MEDIUM

(71) Applicant: PANASONIC INTELLECTUAL PROPERTY MANAGEMENT CO., LTD., Osaka (JP)

(72) Inventors: Yuka YAMAMOTO, Osaka (JP); Nobuaki YABUNOUCHI, Kyoto (JP); Marina ONO, Osaka (JP)

(21) Appl. No.: 17/055,813

(22) PCT Filed: Apr. 19, 2019
(86) PCT No.: PCT/JP2019/016839

§ 371 Date: Nov. 16, 2020

(30) Foreign Application Priority Data
May 24, 2018 (JP) 2018-099685

(51) Int. Cl. A61B 5/16; G06Q 50/20

(52) U.S. Cl. CPC A61B 5/165; A61B 2560/02; G06Q 50/20

(57) ABSTRACT

A behavior induction system includes an obtaining unit which obtains physiological information of a subject and a non-subject, a communicating unit which communicates with a first device which gives a presentation to induce a behavior of the subject and a second device operated by the non-subject to instruct the first device to give the presentation, and a control unit which controls the first device and/or the second device through the communicating unit based on the obtained physiological information of the subject and the non-subject. The control unit includes an evaluating unit which evaluates mental states of the subject and the non-subject based on the obtained physiological information, and a determining unit which determines control content of the first device and/or the second device based on the evaluation result from the evaluating unit. The communicating unit outputs the control content to the first device and/or the second device.

### TECHNICAL FIELD

The present disclosure relates to a behavior induction system, a behavior induction method, and a program which induce a behavior of a person.

### BACKGROUND ART

Accompanied by recent movement of women into the workforce, double-income families with children are increasing. Moreover, accompanied by an increase in nuclear families, household chores and childcare are often performed mainly by women, and impose a heavy load on them. In such circumstances, there is a demand for a reduction in load of childcare imposed on women.

For example, PTL 1 discloses an accident prevention system including a unit which recognizes a risk factor present within a monitoring area, the system detecting an infant or a preschool child (a child up to 6 years old) approaching the risk factor and providing assist information to a caretaker of the infant or preschool child such that the caretaker thereof takes an action to direct the infant or preschool child away from the risk factor.

The traditional technique disclosed in PTL 1 provides the assist information to mothers when their infants or preschool children are approaching risk factors, and can assist mothers to direct their infants or preschool children away from the risk factors or to “teach” their infants or preschool children to recognize those risk factors.

### CITATION LIST

### Patent Literature

PTL 1: Japanese Unexamined Patent Application Publication No. 2004-078304

### SUMMARY OF THE INVENTION

### Technical Problems

However, in the traditional technique disclosed in PTL 1, mothers need to spare their time to teach their infants or preschool children. In particular, mothers in double-income families are heavily burdened with childcare and household chores, and are prone to be stressed.

Accordingly an object of the present disclosure is to provide a behavior induction system, a behavior induction method, and a program which can reduce stress of a non-subject and guide a subject to behave voluntarily.

### Solutions to Problems

The behavior induction system according to one aspect of the present disclosure is a behavior induction system which induces a behavior of a subject, the behavior induction system including: an obtaining unit configured to obtain physiological information of the subject and physiological information of a non-subject; a communicating unit configured to communicate with a first device which gives a presentation to induce the behavior of the subject and a second device operated by the non-subject to instruct the first device to give the presentation; and a control unit configured to control at least one of the first device and the second device through the communicating unit based on the physiological information of the subject obtained and the physiological information of the non-subject obtained. The control unit includes an evaluating unit configured to evaluate a mental state of the subject and a mental state of the non-subject based on the physiological information of the subject obtained and the physiological information of the non-subject obtained; and a determining unit configured to determine at least one of a content of control of the first device and a content of control of the second device based on a result of evaluation by the evaluating unit, and the communicating unit outputs the content of control to at least one of the first device and the second device.

Moreover, the behavior induction method according to one aspect of the present disclosure is a behavior induction method of inducing a behavior of a subject, the method including obtaining physiological information of the subject and physiological information of a non-subject; and controlling at least one of a first device and a second device based on the physiological information of the subject obtained and the physiological information of the non-subject obtained, the first device giving a presentation to induce a behavior of the subject and the second device being operated by the non-subject to give the presentation to the first device. The controlling includes evaluating a mental state of the subject and a mental state of the non-subject based on the physiological information of the subject obtained and the physiological information of the non-subject obtained; determining at least one of a content of control of the first device and a content of control of the second device based on a result of evaluation in the evaluating; and outputting the content of control to at least one of the first device and the second device.

Moreover, the program according to one aspect of the present disclosure causes a computer to execute the behavior induction method.

### Advantageous Effects of Invention

The present disclosure can provide a behavior induction system, a behavior induction method, and a program which can reduce stress of the non-subject and guide the subject to behave voluntarily.

### BRIEF DESCRIPTION OF DRAWINGS

FIG. 1 is a diagram illustrating an outline of the behavior induction system according to an embodiment.

FIG. 2 is a functional block diagram illustrating a configuration of the behavior induction system according to the embodiment.

FIG. 8 is a diagram illustrating a content of control based on the method of evaluating the stress states and the result of evaluation in the embodiment. FIG. 4 is a diagram illustrating a correspondence relation between the result of evaluation of the stress state and the content of control.

FIG. 5 is a flowchart illustrating one example of the behavior induction method according to the embodiment.

### DESCRIPTION OF EXEMPLARY EMBODIMENTS

The embodiment according to the present disclosure will now be specifically described with reference to the drawings.

The embodiments described below illustrate comprehensive or specific examples. Numeral values, shapes, components, arrangements, positions, and connection forms of the components, steps, order of the steps, and the like shown in the embodiments below are exemplary and should not be construed as limitations to the present disclosure. Among the components of the embodiments below, the components not described in an independent claim representing the most superordinate concept of the present disclosure are described as arbitrary components. The drawings are not always strictly drawn. In the drawing, identical referential numerals are given in substantially identical configurations, and the duplication of the description thereof will be omitted or simplified.

### Embodiments

The behavior induction system, the behavior induction method, and the program according to the present embodiment now be described. In the present disclosure, a subject is a target person whose behavior is induced, and a non-subject is a person who induces the behavior of the subject. For example, in the case where the subject is a child including a preschool child, the non-subject is a parent or a teacher. In the case where the subject is an elderly person, the non-subject is a caretaker.

### Outline of Behavior Induction System

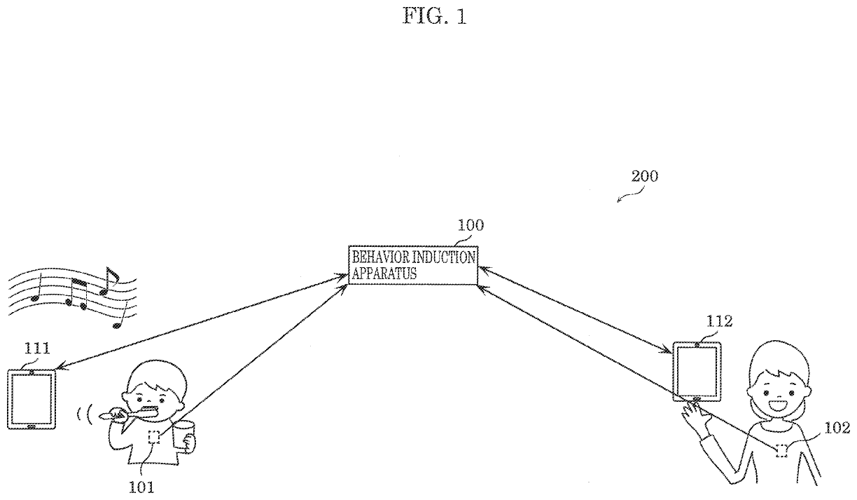

FIG. 1 is a diagram illustrating the outline of behavior induction system 200 according to the present embodiment.

Behavior induction system 200 according to the present embodiment is a system which induces behaviors of a subject. Behavior induction system 200 evaluates the mental states of the subject and the non-subject from their physiological information, and controls at least one of first device 111 and second device 112 based on the result of evaluation. The mental state refers to the state of stress. Thereby behavior induction system 200 can reduce the stress of the non-subject and guide the subject to voluntarily behave.

Behavior induction system 200 includes first sensor 101, second sensor 102, behavior induction apparatus 100, first device 111, and second device 112.

First sensor 101 and second sensor 102 are sensors which measure the physiological information of the subject and that of the non-subject, respectively. The physiological information is physiological information affected by stress, such as heart rate, pulse rate, respiratory rate, blood oxygen saturation, blood pressure, body temperature, and perspiration. Among these examples of the physiological information, the physiological information may be heart rate information, for example, because it is easy to measure. The heart rate information is the information obtained from the heart rate, and is an electrocardiogram, for example. Here, an example will be described where first sensor 101 and second sensor 102 are heart rate sensors.

First device 111 is a device which gives a presentation to induce the behavior of the subject. Here, an example where first device 111 is a tablet terminal will be described.

Second device 112 is a device operated by the non-subject to instruct the first device to give the presentation. Here, an example where second device 112 is a smartphone be described.

The subject is a person or an animal whose behavior is induced. Here, an example where the subject is a preschool child will be described. The non-subject is a person who induces the behavior of the subject. Here, an example where the non-subject is the mother of the preschool child will be described. The behavior refers to a target behavior induced by behavior induction system 200, and includes tidying up, preparation of belongings for the next day, table setting, change of clothes, brushing of teeth, and preparation for sleep. Here, an example where a preschool child is induced to brush teeth will be described.

The presentation to induce the behavior of the subject may be a presentation using a voice or an image to cause the subject to perform a predetermined target behavior.

To instruct the presentation, the non-subject operates second device 112 to input induction information, and transmits the induction information to behavior induction apparatus 100. The induction information refers to the condition related with induction. Here, the induction information includes timing information to induce the behavior of the subject and behavior induction program information indicating an induction method. The timing information refers to a timing at which behavior induction apparatus 100 starts control of at least one of first device 111 and second device 112 based on a predetermined behavior induction program, for example. The behavior induction program information refers to program information to control at least one of first device 111 and second device 112 according to the changes in the mental state of the subject and that of the non-subject.

The outline of behavior induction system 200 according to the present embodiment will now be described with reference to FIG. 1. Behavior induction system 200 is a system which induces a behavior of a preschool child, for example. In behavior induction system 200, for example, at a timing when the preschool child has watched a TV program, behavior induction apparatus 100 controls first device 111 such that first device 111 gives a presentation to induce a behavior of the preschool child, thereby causing the preschool child to notice that it is time to brush teeth and guiding the preschool child to voluntarily brush teeth. The specific example illustrated in FIG. 1 is only one example, and should not be construed as a limitation to behavior induction system 200 according to the present disclosure.

Behavior induction apparatus 100 is connected to first sensor 101, second sensor 102, first device 111, and second device 112. Behavior induction apparatus 100 may be arranged as a stand-alone type, or may be incorporated in an apparatus arranged on a ceiling or a wall, such as a light fixture. To be noted, behavior induction apparatus 100 may be arranged on a server accessible through a network.

The mother can preliminarily input the induction information using second device 112 to perform a setting to operate behavior induction apparatus 100 at a predetermined timing with a predetermined program. The predetermined timing and program will be described later in the section “Configuration of behavior induction system”.

### Configuration of Behavior Induction System

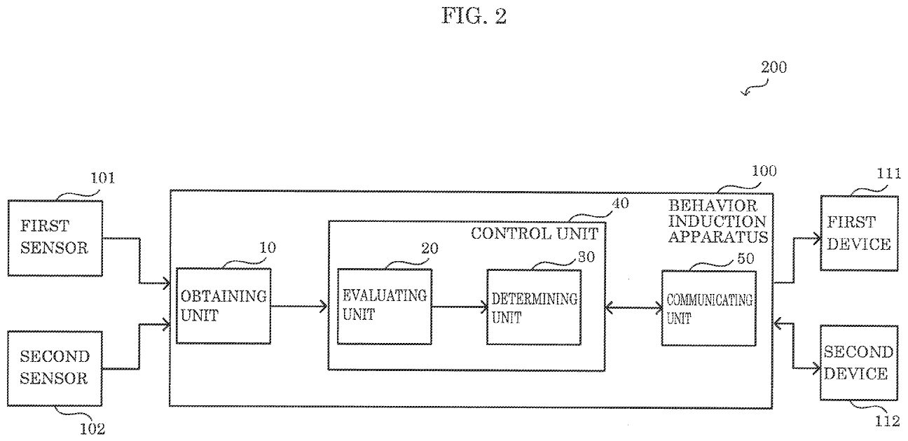

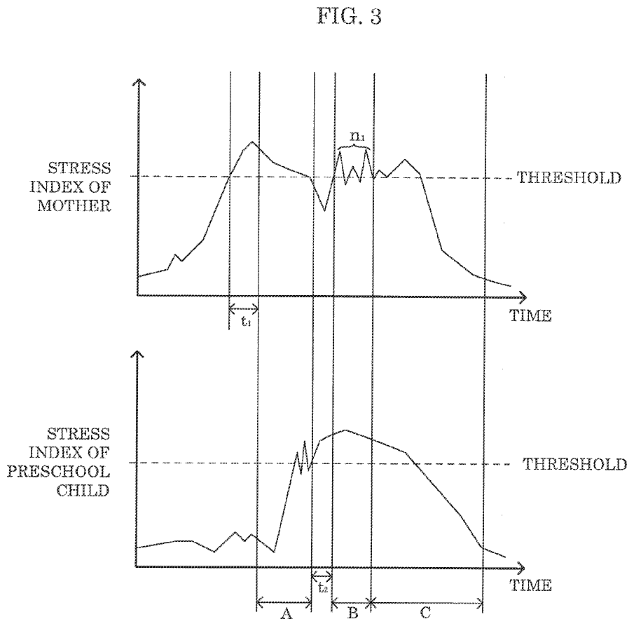

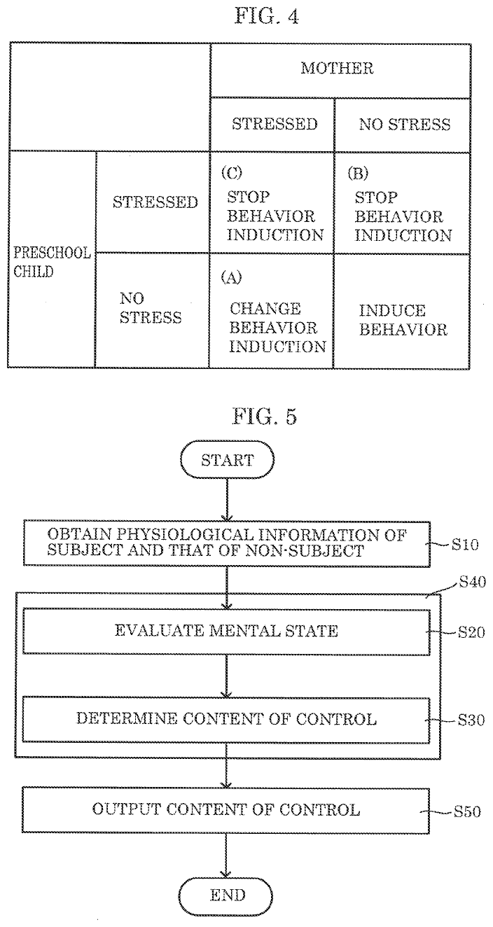

The configuration of behavior induction system 200 according to the present embodiment will now be described. FIG. 2 is a functional block diagram illustrating a configuration of behavior induction system 200 according to the present embodiment.

As illustrated in FIG. 2, behavior induction system 200 includes first sensor 101, second sensor 102, behavior induction apparatus 100, first device 111, and second device 112. Behavior induction apparatus 100 includes obtaining unit 10, control unit 40, and communicating unit 50. Control unit 40 includes evaluating unit 20 and determining unit 30.

First sensor 101 and second sensor 102 are each a sensor which measures the physiological information. First sensor 101 is a sensor which measures the physiological information of the subject, and second sensor 102 is a sensor which measures the physiological information of the non-subject. As described above, the physiological information is the physiological information affected by stress, such as heart rate, pulse rate, respiratory rate, blood oxygen saturation, blood pressure, body temperature, and perspiration. An example in which the physiological information is the heart rate information will be described in the present embodiment, but any other physiological information can be used. For example, the physiological information may be information obtained from the pulse rate (i.e., pulse rate information).

In the case where the physiological information is the heart rate information, first sensor 101 and second sensor 102 are each a sensor (hereinafter, also referred to as heart rate sensor) including a pair of electrodes to be brought into contact of a body surface of a target. The heart rate information obtained by the heart rate sensor is an electric signal obtained from heartbeats, and is an electrocardiogram, for example. The heart rate sensor may be a conductive adhesive gel electrode, or may be a dry electrode made of conductive fibers. The site for the heart rate sensor to be mounted on is a breast. The heart rate sensor is in the form of a wear including electrodes integrally formed with the wear. The wear is an undershirt or an underwear, for example.

In the case where the physiological information is the pulse rate information, first sensor 101 and second sensor 102 are each a sensor (hereinafter, also referred to as pulse rate sensor) which measures a change in blood amount in the blood vessel with a phototransistor and a photodiode by way of reflected light or transmitted light, for example. The site for the pulse rate sensor to be mounted on is a wrist, an ankle, a finger, or an upper arm. The pulse rate sensor is in the form of a band (such as a watch), an attachment applied to a neck or the like, or a pair of glasses, for example. Alternatively, the pulse rate sensor may be an image sensor which measures the pulse rate information from a change in chromaticity of the skin of a face or a hand to calculate the pulse rate.

In the case where the physiological information is the respiratory rate, for example, first sensor 101 and second sensor 102 are each a belt type sensor including a pressure sensor wrapped around the breast or the abdomen, or a temperature sensor attached to under the nose.

In the case where the physiological information is the blood oxygen saturation, the biosensor is a sensor which measures a change in saturated oxygen concentration in the blood in the blood vessel with a phototransistor and two types of photodiodes by way of reflected light or transmitted light, for example.

In the case where the physiological information is the blood pressure, the biosensor is, for example, a sensor including a belt with a pressure sensor, the belt, for use, being wrapped around an upper arm and a finger tip or a radial bone.

In the case where the physiological information is the body temperature, the biosensor is a thermocouple sensor attached to the site where the capillary vessels are likely to contract due to stress, such as a palm or a nose tip, for example.

In the case where the physiological information is perspiration, the biosensor is a sensor including a pair of detection electrodes brought into contact with the site where perspiration is likely to occur, such as a palm or a face, for example.

In addition to any of these sensors above, first sensor 101 and second sensor 102 may further include an accelerometer which detects the movement of the preschool child and that of the mother. For example, in the case where the preschool child performs a movement such that the heart rate information thereof is changed, by way of first sensor 101 including the heart rate sensor and the accelerometer, the mental state of the preschool child during the period in which the preschool child performed such a movement can be excluded from the target for the evaluation of the mental state. Thereby behavior induction apparatus 100 can distinguish the variation in physiological information caused by the movement of the preschool child from a variation in physiological information caused by any psychological factor. For this reason, behavior induction apparatus 100 can more accurately evaluate the mental state of the preschool child and that of the mother, and thus can more appropriately control first device 111 and second device 112. In other words, behavior induction apparatus 100 can more accurately grasp the stress states of the preschool child and the mother, and can more appropriately guide the behavior of the preschool child, thus reducing the stress of the mother.

As described above, first sensor 101 and second sensor 102 measure the physiological information of the preschool child and that of the mother, respectively, and transmit these pieces of physiological information to behavior induction apparatus 100.

Behavior induction apparatus 100 obtains the physiological information of the preschool child and that of the mother transmitted from first sensor 101 and second sensor 102, respectively, and evaluates the mental state of the preschool child and that of the mother from the heart rate information obtained. Based on the result of evaluation of the mental states, behavior induction apparatus 100 controls at least one of first device 111 and second device 112.

Behavior induction apparatus 100 includes obtaining unit 10, control unit 40, and communicating unit 50. Control unit 40 includes evaluating unit 20 and determining unit 30. Behavior induction apparatus 100 may be installed on a ceiling or a wall, or may be incorporated in an apparatus installed in a ceiling or a wall. The apparatus installed on a ceiling or a wall may be incorporated in a light fixture or a behavior detecting unit (such as a camera). As described above, behavior induction apparatus 100 may be arranged on a server connected through the Internet.

Obtaining unit 10 obtains the heart rate information of the preschool child and that of the mother transmitted from first sensor 101 and second sensor 102, and transmits these obtained pieces of physiological information to evaluating unit 20. Obtaining unit 10 is a wireless communication interface according to Bluetooth (registered trademark), IrDA, or Wi-Fi (registered trademark).

Control unit 40 controls behavior induction apparatus 100 based on the induction information obtained by communicating unit 50. Control unit 40 includes a central processing unit (CPU), a read only memory (ROM) which stores programs, a read access memory (RAM), evaluating unit 20, and determining unit 30. Evaluating unit 20 and determining unit 30 are implemented through execution of the programs by the CPU.

Control unit 40 controls at least one of first device 111 and second device 112 based on the physiological information of the preschool child and that of the mother. Specifically in control unit 40, evaluating unit 20 obtains the heart rate information of the preschool child and that of the mother transmitted from obtaining unit 10, evaluates the mental states of the preschool child and the mother from the obtained heart rate information, and transmits the result of evaluation to determining unit 30. The heart rate information is an electrocardiogram, for example. Evaluating unit 20 calculates the indices such as the heart rate and the heart rate variability from the electrocardiograms of the preschool child and the mother. The heart rate is calculated by multiplying the inverse number of the heart rate interval (R-R interval, RRI) by 60, where the heart rate interval is the interval between two successive R waves in the electrocardiogram. The heart rate variability is a coefficient of variation in variation of the heart rate (Coefficient of Variation of R-R intervals, CvRR) and the frequency components in variation of the heart rate, for example. The frequency components in variation of the heart rate are a high frequency (HF), which is the integrated value of a power spectrum of a high frequency domain from 0.14 Hz to 0.4 Hz, and a low frequency (LF), which is the integrated value of a power spectrum of a low frequency domain from 0.04 Hz to 0.14 Hz, for example. These frequency components in variation of the heart rate can be determined by frequency analysis of the RRI using fast Fourier transform.

Using one or more of the indices above, evaluating unit 20 evaluates the stress states of the preschool child and the mother. The content of control based on the method of evaluating the stress states and the result of evaluation will now be described.

FIG. 3 is a diagram illustrating the content of control based on the method of evaluating the stress states and the result of evaluation according to the present embodiment.

Evaluating unit 20 calculates the stress indices of the preschool child and the mother using one or more of the indices above, for example. The stress index may be calculated according to the change rate of the index used in the evaluation, or the value of the index may be used as it is.

In behavior induction system 200 according to the present embodiment, evaluating unit 20 calculates a change over time in stress index indicating the mental state based on the heart rate information, in the case where the value of the stress index is higher than the threshold for a predetermined time or the predetermined number of times, evaluating unit 20 outputs an evaluation result that the mental state represents a stressed state. For example, as illustrated in FIG. 3, in the case where the value of the stress index of mother is higher than the threshold for predetermined time t₁, evaluating unit 20 outputs an evaluation result that the mental state of the mother represents a stressed state. In the case where the value of the stress index of the preschool child is higher than the threshold for predetermined time t₂, evaluating unit 20 outputs an evaluation result that the mental state of the preschool child represents a stressed state. In the case where the value of the stress index of the mother is higher than the threshold the predetermined number of times (e.g., n₁ times), evaluating unit 20 outputs an evaluation result that the mental state of the mother represents a stressed state. Thereby, for example, an increase in stress index derived not from stress, such as a change in physiological information caused by a movement, can be excluded from the determination of the mental state, resulting in accurate evaluation of the stress state.

The threshold may be a preset value, or may be a value calculated through a test for setting the threshold. The threshold for the preschool child and that for the mother may be the same or different.

Determining unit 30 determines at least one of the content of control of first device 111 and that of second device 112 based on the result of evaluation by evaluating unit 20. Hereinafter, the operation of determining unit 30 will be described by way of a specific example. FIG. 4 is a diagram illustrating a correspondence relation between the result of evaluation of the stress states and the content of control.

As illustrated in “Induce behavior” in FIG. 4, in the case where evaluating unit 20 outputs an evaluation result that the preschool child and the mother are not stressed, determining unit 30 controls first device 111 based on the induction information preliminarily input by the mother. At this time, first device 111 gives a presentation to cause the preschool child to notice that it is time to perform a predetermined target behavior. Examples of the presentation include reproduction of different tunes for different target behaviors, or display of a moving picture in which a character says, “It's time to tidy up!” or the like and performs the target behavior (such as tidying up). Then, first device 111 gives a presentation to cause the preschool child to perform the predetermined target behavior. Examples of the presentation include reproduction of different tunes for different target behaviors, or display of a moving picture in which a character is singing a song and performing the target behavior.

As illustrated in (A) of FIG. 4, in the case where evaluating unit 20 outputs an evaluation result that at least the mother is in a stressed state, determining unit 30 changes at least one of the content of control of first device 111 and that of second device 112. For example, in the case where the content of control of first device 111 is changed, examples thereof include an increase in reproduction speed of the music, or display of a moving picture in which a character says, “Who will win?”, and competes with the preschool child for how quickly the target behavior is completed. For example, in the case where the content of control of second device 112 is changed, a presentation to reduce the stress of the mother is given. At this time, for example, second device 112 presents an image showing that the preschool child is doing the target behavior, and gives an advice that “He/she is working hard. So, wait for a while”; or presents the state of accomplishment of the target behavior by the preschool child, and suggests a change of the content of control of first device 111 according to the state of accomplishment. Thereby, the mother can take a cue to change her mood, reducing her stress.

Here, in addition to the evaluation of the mental states of the subject and the non-subject, evaluating unit 20 may further evaluate whether the subject has performed a predetermined target behavior. The evaluation whether the subject has performed the predetermined target behavior also includes evaluation how much the subject has done the predetermined target behavior.

In the case where evaluating unit 20 outputs an evaluation result that the preschool child has not performed the target behavior yet and the mother is in a stressed state ((A) of FIG. 4), determining unit 30 changes at least the content of control of first device 111 to a control to raise a motivation of the preschool child to perform the target behavior. A presentation to raise the motivation of the preschool child to perform the target behavior may be given with at least one of the voice and the image. In the case where the presentation is given with both of the voice and the image, examples of such a presentation include reproduction of the preschool child's favorite music, display of a moving picture in which a character is doing the target behavior, or display of a moving picture in which the character says to the preschool child, “Let's do it together”, “Great!”, “Good job!” or the like, and cheerfully performs the target behavior. Thereby, the mother can take a cue to change her mood, readily reducing her stress. For this reason, the mother can relatively calmly treat the preschool child even in a situation where the mother gives some support for behavior induction of the preschool child, for example. Owing to the presentation to raise the motivation to perform the target behavior, the preschool child readily performs the target behavior. At this time, the preschool child can more willingly perform the target behavior, for example, in response to the support calmly offered by the mother.

As illustrated in (B) and (C) of FIG. 4, in the case where evaluating unit 20 outputs an evaluation result that the preschool child is in a stressed state or the preschool child and the mother both are in stressed states, determining unit 30 changes at least the content of control of first device 111 to a control to give a presentation to stop the behavior induction of the preschool child. Examples of the presentation to stop the behavior induction of the preschool child include stop of the music by the preschool child, or display of a moving picture in which the character says “Bye!” with waving, and goes home. This enables individual timeout of the preschool child and the mother. This can avoid forcibly continued behavior induction of the preschool child by the mother in the case where the mother is in a stressed state, thus reducing experiences such that the preschool child and the mother have negative feelings to the behavior induction. In the case where the preschool child is in a stressed state, the behavior induction, if tried, is often unsuccessful. By stopping the behavior induction once, the mental state of the preschool child can be calmed. Thereby, the preschool child can change the mood and work when the preschool child starts the next behavior. Thus, the behavior of the preschool child can be effectively induced.

When having changed the content of control of first device 111 to the control to give a presentation to stop the behavior induction of the preschool child, determining unit 30 changes the content of control of second device 112 to a control to notify the mother of the stop of the behavior induction. The notification may be performed only with an image, or may be performed with an image and a voice. Thereby, the mother can know that the behavior induction of the preschool child has been stopped, and thus can change her mood for the next behavior induction. This enables individual timeout of the preschool child and the mother, thus facilitating charge of their moods for the next behavior.

First device 111 gives a presentation to induce a behavior of the preschool child. Here, the presentation to induce a behavior of the preschool child includes not only the presentation to cause the preschool child to notice that it is time to perform the target behavior, but also the presentation to raise the motivation of the preschool child to perform the target behavior and the presentation to stop the behavior induction of the preschool child. The presentation may be a presentation with a voice, may be a presentation with an image, or may be a presentation with a voice and an image.

The mother operates second device 112 to instruct first device 111 to give a presentation. For example, second device 112 transmits the induction information to behavior induction apparatus 100. Second device 112 may also transmit a change in the content of control of first device 111 according to the state of accomplishment of the target behavior by the preschool child to behavior induction apparatus 100.

Examples of first device 111 and second device 112 include smartphones, tablet terminals, wearable terminals, smart speakers, and personal computers. First device 111 and second device 112 each include a presenting unit (not illustrated), an inputting unit (not illustrated), and a communicating unit (not illustrated). For example, the presenting unit is a speaker for a presentation given with a voice, and is a liquid crystal display or an organic electroluminescent (EL) display for a presentation given with an image. The inputting unit is, for example, a touch panel, a keyboard, a mouse, or a microphone. First device 111 and second device 112 are connected to behavior induction apparatus 100 through communicating unit 50. Communicating unit 50 is a wireless communication interface according to Bluetooth (registered trademark), IrDA, or Wi-Fi (registered trademark).

Behavior induction system 200 may include a behavior detecting unit (not illustrated). The behavior detecting unit detects a behavior of a person, and transmits behavior information indicating a behavior to behavior induction apparatus 100. In the detection of a person, the behavior of the person him/herself may be detected, or a movement of an object caused by a movement of the person may be detected. In other words, the behavior information may be information indicating a movement of a person, or may be information indicating a movement of an object caused by a movement of the person.

The behavior detecting unit is a camera or a sensor, for example. in the case where the behavior detecting unit is a camera, the behavior detecting unit may detect a movement of the preschool child tidying up toys or picture books, or may detect movements of toys or picture books which the preschool child is tidying up, where the toys or the picture books are tagged. In the case where the behavior detecting unit is a sensor, the behavior detecting unit may detect the movement of each toy or picture book which the preschool child is tidying up, by measuring a change in weight with a weight sensor integrated in a container such as a toy box. Alternatively, the behavior detecting unit may detect a movement of the preschool child brushing teeth or changing the cloth, for example.

The behavior detecting unit may be wirelessly or wiredly connected to behavior induction apparatus 100. The behavior detecting unit may be arranged as a stand-alone type, or may be incorporated in an apparatus arranged on a ceiling or a wall, such as a light fixture.

In the case where first device 111 includes a camera, the movement of the preschool child may be detected with the camera in first device 111.

Behavior induction apparatus 100 may transmit the information on the movement of the preschool child detected by the behavior detecting unit or the camera in first device 111 to second device 112. Thereby, the mother can check the movement of the preschool child with a monitor in second device 112, and thus can dearly know the progress of the target behavior (such as tidying up or brushing of teeth) of the preschool child. For this reason, the mother readily predicts the time until the preschool child accomplishes the target behavior and whether the support is needed. Accordingly, the stress of the mother can be reduced, thus reducing emotional attitudes of the mother toward the preschool child.

### Behavior Induction Method

Next, the behavior induction method according to the present embodiment will be described. The behavior induction method according to the present embodiment is executed by the operations of the configurations in behavior induction apparatus 100. FIG. 5 is a flowchart illustrating one example of the behavior induction method according to the present embodiment.

In the behavior induction method according to the present embodiment, first, obtaining unit 10 obtains physiological information of the preschool child and the physiological information of the mother from first sensor 101 and second sensor 102, respectively (obtaining step S10). In the next step, in behavior induction apparatus 100, control unit 40 controls at least one of first device 111 and second device 112 based on the obtained physiological information of the preschool child and the obtained physiological information of the mother (control step S40). Specifically, in control step S40, evaluating unit 20 evaluates the mental states of the preschool child and the mother based on the obtained pieces of physiological information, respectively (evaluation step S20). Here, the mental state represents a stress state. In the next step, in control step S40, determining unit 30 determines at least one of the content of control of first device 111 and that of second device 112 based on the result of evaluation in evaluation step S20 (determination step S30).

In the next step, communicating unit 50 outputs the content of control to at least one of first device 111 and second device 112 (output step S50).

Hereinafter, details of the processing in control step S40 will be described, again, with reference to FIG. 4.

As illustrated in FIG. 4, in the case where evaluating unit 20 outputs an evaluation result that the preschool child and the mother both are not stressed in evaluation step S20, based on the induction information preliminarily input by the mother, determining unit 30 determines at least one of the content of control of first device 111 and that of second device 112 in determination step S30.

In the case where in evaluation step S20, evaluating unit 20 outputs an evaluation result that the preschool child is not stressed and the mother is in a stressed state, determining unit 30 changes at least one of the content of control of first device 111 and that of second device 112 in determination step S30. At this time, in evaluation step S20, evaluating unit 20 further evaluates whether the preschool child has performed the predetermined target behavior (such as tidying up). In the case where evaluating unit 20 outputs an evaluation result that the preschool child has not performed the predetermined target behavior yet, in determination step S30, determining unit 30 changes at least the content of control of first device 111 to the control to give the presentation to raise the motivation of the preschool child to perform the target behavior.

In the case where in evaluation step S20, evaluating unit 20 outputs an evaluation result that the preschool child is in a stressed state or that the preschool child and the mother both are in stress states, in determination step S30, determining unit 30 changes the content of control of first device 111 to the control to stop the behavior induction. At this time, determining unit 30 changes the content of control of second device 112 to the control to notify the mother of the stop of the behavior induction.

The presentations above are as described above in the description of the operational example of determining unit 30, and therefore the description thereof will be omitted here.

It should be noted that in evaluation step S20, after a predetermined time has passed from the start of the behavior induction program, control unit 40 may obtain the behavior information of the preschool child through communicating unit 50 from the behavior detecting unit (not illustrated) or the camera in first device 111. At this time, control unit 40 may obtain the behavior information by requesting the behavior detecting unit or the camera in first device 111 for the behavior information or without requesting the behavior detecting unit for the behavior information. Evaluating unit 20 evaluates the state of accomplishment of the target behavior by the preschool child based on the behavior information obtained by communicating unit 50. Determining unit 30 changes the content of control of first device 111 based on the state of accomplishment of the target behavior by the preschool child. For example, in the case where the behavior of the preschool child is induced by reproducing the music, determining unit 30 may control the reproduction speed of the music to raise the motivation of the preschool child. The time may be controlled by repeatedly reproducing at least part of the music, such that the music is over at the time when the target behavior of the preschool child is accomplished. Thereby the preschool child can complete the behavior during the period in which the music is reproduced, and thus can have a feel of accomplishment.

### Operational Example of Behavior Induction System

Next, the operation of behavior induction system 200 according to the present embodiment will be described.

Here, the subject is the preschool child, and the target behavior to be induced is tidying up of toys or the like. First device 111 is tablet terminal 111, second device 112 is smartphone 112 of the preschool child's mother, and the behavior detecting unit is a camera.

Although not illustrated, in behavior induction system 200, first, using smartphone 112, the mother preliminarily selects the timing information such as the time to start behavior induction and the program information to cause tablet terminal 111 to execute a presentation for inducing the behavior of the preschool child, and transmits these as induction information to behavior induction apparatus 100. For example, the mother can preliminarily set the induction information for inducing the behavior of the preschool child, for example, select “19:00” as the timing information and “reproduction of the preschool child's favorite music for 10 minutes” as the program information. The induction information may be set every time when execution of the behavior induction of the preschool child is desired, or may be periodically repeated by setting the day of the week and the time.

After obtaining the induction information from smartphone 112, behavior induction apparatus 100 starts the behavior induction at a predetermined timing based on the induction information. At this time, control unit 40 transmits a command to cause evaluating unit 20 to evaluate the mental states of the preschool child and the mother. At this time, control unit 40 may request the camera for picture data indicating the current situation. Thereby as the degree of mess, for example, the initial situation of the room when the behavior induction program is started can be compared to the situation of the progress of tidying up after a predetermined time has passed. Thus, the time needed for accomplishment of the behavior can be more accurately predicted.

Whether the behavior induction program is executed or not, first sensor 101 and second sensor 102 may measure the physiological information (here, the heart rate information) of the preschool child and that of the mother, respectively, and transmit these pieces of physiological information to obtaining unit 10 in behavior induction apparatus 100. Evaluating unit 20 may evaluate the mental states from the physiological information of the preschool child and that of the mother through obtaining unit 10, and update the thresholds of the stress indices for every predetermined period. Thereby, the stress state can be evaluated according to the seasonal or daily health condition, and the behavior induction can be stably continued.

When the predetermined time has passed after the execution of the behavior induction program, control unit 40 requests the camera for the picture data indicating the progress of tidying up. When the camera transmits the picture data indicating the progress to control unit 40, evaluating unit 20 calculates the time until accomplishment of the behavior. The term “accomplishment of the behavior” refers to completion of an induced behavior, and means the completion of tidying up here. At this time, from the obtained picture data, evaluating unit 20 calculates the progress, that is, the degree of mess after the predetermined time has passed from the start of tidying up. Evaluating unit 20 compares the degree of mess after the predetermined time has passed to the initial situation when the behavior induction program is started, i.e., the degree of mess when the tidying up is started, and calculates the time until the completion of tidying up. Here, for example, the degree of mess is calculated as follows. Toys and picture books are tagged, and the situation of the room are captured with the camera. The number of tags present in the room is identified from the captured image, followed by calculation of the number of tags in the room when tidying up is started and the number of tags after a predetermined time has passed from the start of tidying up. Then, the difference therebetween is calculated, and is divided by the time which has passed. Thereby the speed of tidying up by the preschool child is calculated. From the calculated speed of tidying up, the time until the completion of tidying up is calculated. Considering a possibility that the tidying-up speed may change, a predetermined time is added to the calculated time until the completion of tidying up to calculate the time until the completion of tidying up. In response to an instruction from smartphone 112, control unit 40 may cause smartphone 112 to present the time until the completion of tidying up. Thereby the mother can know the time until the completion of tidying up, thus reducing the stress.

Determining unit 30 changes the behavior induction program according to the states of stress of the preschool child and the mother. For example, in the case where evaluating unit 20 outputs an evaluation result that the preschool child and the mother both are not stressed, evaluating unit 20 calculates the difference between the calculated time until the completion of tidying up and the time set by the behavior induction program (10 minutes). Determining unit 30 determines the reproduction pattern of the music according to the time length of the difference calculated in evaluating unit 20. The reproduction pattern of the music may be a pattern in which the reproduction speed of the music is reduced, or may be a pattern in which part of the music, such as the chorus part of the music is repeated. It is preferred that such an adjustment to extend the preset time (10 minutes) be performed such that the preschool child does not know the adjustment. The reproduction pattern of music may be appropriately determined according to the time length of the difference.

For example, in the case where evaluating unit 20 outputs an evaluation result that the mother is in a stressed state, determining unit 30 changes the control to a control to adjust to increase the reproduction speed of the music. It is preferred that the reproduction speed be adjusted to a speed such that the preschool child can behave to the speed of the music.

For example, in the case where evaluating unit 20 outputs an evaluation result that at least the preschool child is in a stressed state, determining unit 30 changes the control to the control to stop the behavior induction program. In other words, the reproduction of the music is stopped. Then, determining unit 30 transmits a notice to the mother that the behavior induction program has been stopped.

Behavior induction apparatus 100 transmits a command based on this determination to first device 111 and smartphone 112.

By the behavior induction method above, the mother can reduce the stress, and the preschool child can voluntarily perform the target behavior. In the case where it is determined that the preschool child and the mother both are in stress states, by stopping the behavior induction, the preschool child and the mother can calm themselves, and can afford self-examination about that the accomplishment of the target behavior is unsuccessful. Emotional attitudes of the mother toward the preschool child can also be reduced, and thus the preschool child can have a stronger motivation to accomplish the target behavior. Accordingly, when a presentation for behavior induction is given on first device 111 (for example, a predetermined tune is reproduced), the preschool child notices that it is time to tidy up, and is willing to tidy up.

As described above, behavior induction system 200 according to the present embodiment is a behavior induction system which induces a behavior of a subject, and includes obtaining unit 10 which obtains physiological information of the subject and physiological information of a non-subject, first device 111 which gives a presentation to induce the behavior of the subject, communicating unit 50 which communicates with second device 112 operated by the non-subject to give the presentation to first device 111, and control unit 40 which controls at least one of first device 111 and second device 112 through communicating unit 50 based on the obtained physiological information of the subject and the obtained physiological information of the non-subject. Control unit 40 includes evaluating unit 20 which evaluates mental states of the subject and the non-subject based on the obtained physiological information thereof, and determining unit 30 which determines at least one of the content of control of first device 111 and that of second device 112 based on the result of evaluation by evaluating unit 20. Communicating unit 50 outputs the content of control to at least one of first device 111 and second device 112.

Thus, by evaluating the mental states of the subject and the non-subject from the physiological information of the subject and that of the non-subject, behavior induction can be performed according to the mental states of the subject and the non-subject. This can reduce the mental loads on the subject and the non-subject. In particular, due to reduced stress of the non-subject, the frequency of forced behavior induction of the subject by the non-subject is decreased. For this reason, the subject hardly has a negative feeling about induction of behaviors, and can voluntarily behave. Accordingly the stresses of the non-subject and the subject caused by the behavior induction can be reduced by the behavior induction system according to the present disclosure. As a result, the subject can voluntarily perform the target behavior.

Moreover, in behavior induction system 200 according to the present embodiment, evaluating unit 20 may calculate a change over time in stress index indicating the mental state based on the physiological information, and may evaluate the mental state as a stress state in the case where the value of the stress index is higher than the threshold for the predetermined time or the predetermined number of times.

Thereby, for example, an increase in stress index derived not from stress, such as a change in physiological information caused by a movement, can be excluded from the determination of the mental state, resulting in accurate evaluation of the stress state.

In behavior induction system 200 according to the present embodiment, in the case where evaluating unit 20 outputs an evaluation result that at least the non-subject is in a stressed state, determining unit 30 may change at least one of the content of control of first device 111 and that of second device 112.

Thereby, the non-subject can take a cue to change the mood, reducing the stress.

In behavior induction system 200 according to the present embodiment, further, evaluating unit 20 may evaluate whether the subject has performed a predetermined target behavior, and in the case where evaluating unit 20 outputs an evaluation result that the subject has not performed the target behavior and the non-subject is in a stressed state, determining unit 30 may change the content of control of at least first device 111 to a control to give the presentation to raise the motivation of the subject to perform the target behavior.

Thereby, the non-subject can take a cue to change the mood, reducing the stress. For this reason, for example, the non-subject can relatively calmly treat the subject even in a situation where the non-subject gives some support for the behavior induction of the subject. Owing to the presentation to raise the motivation to perform the target behavior, the subject readily performs the target behavior. At this time, the subject can more willingly perform the target behavior, for example, in response to the support calmly offered by the non-subject.

In behavior induction system 200 according to the present embodiment, in the case where evaluating unit 20 outputs an evaluation result that the subject is in a stressed state or the subject and the non-subject both are in stressed states, determining unit 30 may change the content of control of at least first device 111 to a control to give a presentation to stop the behavior induction of the subject.

This enables individual timeout of the subject and the non-subject. This can avoid forcibly continued behavior induction of the subject by the non-subject in the case where the non-subject is in a stressed state, thus reducing experiences such that the subject and the non-subject have negative feelings to the behavior induction. In the case where the subject is in a stressed state, the behavior induction, if tried, is often unsuccessful. By stopping the behavior induction once, the mental state of the subject can be calmed. Thereby, the subject can change the mood and work when the subject starts the next behavior. Thus, the behavior of the subject can be effectively induced.

In behavior induction system 200 according to the present embodiment, when having changed the content of control of first device 111 to the control to give the presentation to stop the behavior induction of the subject, determining unit 30 may change the content of control of second device 112 to a control to notify the non-subject of the stop of the behavior induction.

Thereby, the non-subject can know that the behavior induction of the subject has been stopped, and thus can change the mood for the next behavior induction. This enables individual timeout of the subject and the non-subject, thus facilitating change of their moods for the next behavior.

The behavior induction method according to the present embodiment is a behavior induction method of inducing a behavior of a subject, and includes obtaining physiological information of the subject and physiological information of a non-subject, and controlling at least one of first device 111 which gives a presentation to induce the behavior of the subject and second device 112 operated by the non-subject to give the presentation on first device 111, based on the obtained physiological information of the subject and the obtained physiological information of the non-subject. The controlling includes evaluating mental states of the subject and the non-subject based on the obtained physiological information, determining at least one of the content of control of first device 111 and that of second device 112 based on the result of evaluation in the evaluating, and outputting the content of control to at least one of first device 111 and second device 112.

Thus, the behavior induction method according to the present disclosure includes evaluating the mental states of the subject and the non-subject from the physiological information of the subject and the non-subject, which enables the behavior induction according to the mental states of the subject and the non-subject. This can reduce the mental loads on the subject and the non-subject. In particular, due to reduced stress of the non-subject, the frequency of forced behavior induction of the subject by the non-subject is decreased. For this reason, the subject hardly has a negative feeling about induction of behaviors, and can voluntarily behave. Accordingly, the stresses of the non-subject and the subject caused by the behavior induction can be reduced by the behavior induction system according to the present disclosure. As a result, the subject can voluntarily perform the target behavior.

These comprehensive or specific aspects may be implemented with systems, methods, integrated circuits, computer programs, or recording media such as computer-readable CD-ROMs, or may be implemented with any combination of systems, methods, integrated circuits, computer programs, and recording media.

Although control unit 40 has been implemented with a software program in the embodiments above, control unit 40 may be implemented with dedicated hardware.

Although the behavior induction system, the behavior induction method, and the program according to the present disclosure have been described based on the embodiments above, these embodiments should not be construed as limitations to the present disclosure. The present disclosure also covers a variety of modifications of the embodiments conceived by persons skilled in the art the present disclosure without departing from the gist of the present disclosure, and other embodiments including combinations made of part of the components in the embodiments.

Although an example in which first device 111 and second device 112 are a tablet terminal and a smartphone, respectively, in the present embodiment, first device 111 and second device 112 may be a wearable terminal in the form of a watch, for example. Although an example in which first sensor 101 and second sensor 102 are heart rate sensors has been described in the present embodiment, these ray be pulse rate sensors. The pulse rate sensor may be integrated with a wearable terminal in the form of a watch, for example. The wearable terminal in the form of a watch may include a presenting unit such as a display and a speaker, an inputting unit such as a touch panel and a microphone, and a camera.

Thereby, the subject and the non-subject may easily and comfortably perform the behavior induction in the daily life.

Although an example in which the subject subjected to the behavior induction and the non-subject instructing the behavior induction are each a single person in the present embodiment, each of them may be a group of people. In this case, management may be performed using a terminals, such as a computer, which can collectively manage a plurality of first sensors and a plurality of second sensors and the information of the plurality of first sensors and that of the plurality of second sensor.

This can reduce the stress of the non-subject (here, a caretaker) even in institutes such as nursery schools and retirement homes, and more appropriately guide the subject to accomplish the target behavior.

### REFERENCE MARKS IN THE DRAWINGS

10 obtaining unit

20 evaluating unit

30 determining unit

40 control unit

50 communicating unit

100 behavior induction apparatus

101 first sensor

102 second sensor

111 first device (tablet terminal)

112 second device (smartphone)

200 behavior induction system

1. A behavior induction system which induces a behavior of a subject, the behavior induction system comprising:
an obtaining unit configured to obtain physiological information of the subject and physiological information of a non-subject;
a communicating unit configured to communicate with a first device which gives a presentation to induce the behavior of the subject and a second device operated by the non-subject to instruct the first device to give the presentation; and
a control unit configured to control at least one of the first device and the second device through the communicating unit based on the physiological information of the subject obtained and the physiological information of the non-subject obtained,
wherein the control unit includes:
an evaluating unit configured to evaluate a mental state of the subject and a mental state of the non-subject based on the physiological information of the subject obtained and the physiological information of the non-subject obtained; and
a determining unit configured to determine at least one of a content of control of the first device and a content of control of the second device based on a result of evaluation by the evaluating unit, and
the communicating unit outputs the content of control to at least one of the first device and the second device.

2. The behavior induction system according to claim 1,
wherein based on the physiological information, the evaluating unit calculates a change over time of a stress index indicating the mental state, and outputs an evaluation result that the mental state represents a stressed state when a value of the stress index is larger than a threshold for a predetermined time or a predetermined number of times.

3. The behavior induction system according to claim 2,
wherein when the evaluating unit outputs an evaluation result that at least the non-subject is in a stressed state, the determining unit changes at least one of the content of control of the first device and the content of control of the second device.

4. The behavior induction system according to claim 2,
wherein the evaluating unit further evaluates whether the subject has performed a predetermined target behavior, and
when the evaluating unit outputs an evaluation result that the subject has not performed the target behavior and the non-subject is in a stressed state, the determining unit changes at least the content of control of the first device to a control to give a presentation to raise a motivation of the subject to perform the target behavior.

5. The behavior induction system according to claim 2,
wherein when the evaluating unit outputs an evaluation result that the subject is in a stressed state, the determining unit changes at least the content of control of the first device to a control to give a presentation to stop behavior induction of the subject.

6. The behavior induction system according to claim 5,
wherein when the content of control of the first device is changed to the control to give the presentation to stop the behavior induction of the subject, the determining unit changes the content of control of the second device to a control to notify the non-subject of stop of the behavior induction.

7. A behavior induction method of inducing a behavior of a subject, the method comprising:
obtaining physiological information of the subject and physiological information of a non-subject; and
at least one of a first device and a second device based on the physiological information of the subject obtained and the physiological information of the non-subject obtained, the first device giving a presentation to induce a behavior of the subject and the second device being operated by the non-subject to give the presentation to the first device,
wherein the controlling includes:
evaluating a mental state of the subject and a mental state of the non-subject based on the physiological information of the subject obtained and the physiological information of the non-subject obtained;
determining at least one of a content of control of the first device and a content of control of the second device based on a result of evaluation in the evaluating; and
outputting the content of control to at least one of the first device and the second device.

8. A non-transitory computer-readable recording medium for use in a computer, the recording medium having a computer program recorded thereon for causing the computer to execute the behavior induction method according to claim 7.
